(19) United States
(12) Patent Application Publication
(10) Pub. No.: US 2019/0234926 A1
(43) Pub. Date: Aug. 1, 2019

(54) METHOD OF PREDICTING FLAVOR PERFORMANCE

(71) Applicant: McCormick & Company, Incorporated, Hunt Valley, MD (US)

(72) Inventors: Dmitriy ZASYPKIN, Cockeysville, MD (US); Michael MADSEN, Baltimore, MD (US)

(73) Assignee: McCormick & Company, Incorporated, Hunt Valley, MD (US)

(21) Appl. No.: 16/315,361

(22) PCT Filed: Jul. 5, 2017
(86) PCT No.: PCT/US2017/040706

§ 371 Date: Jan. 4, 2019

Related U.S. Application Data
(60) Provisional application No. 62/358,756, filed on Jul. 6, 2016.

(51) Int. Cl. G01N 33/14; G01N 27/06

(52) U.S. Cl. CPC G01N 33/14; A23L 27/72; G01N 27/06

(57) ABSTRACT

A method of measuring flavor properties. A method of quantifying, measuring, and/or predicting the compatibility, solubility, effective encapsulation and/or emulsification of flavors in a carrier, and performance in applications is described, by measuring the polarity of the flavor through its dielectric constant.

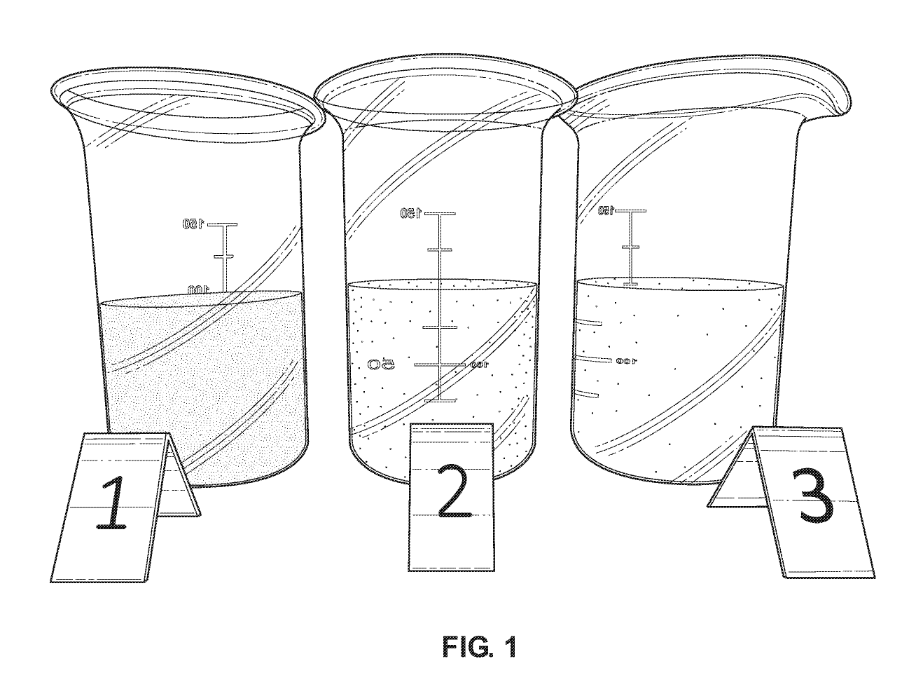

### CROSS-REFERENCE TO RELATED APPLICATION

The instant application claims priority to U.S. Provisional Application No. 62/358,756 filed Jul. 6, 2016, the disclosure of which is expressly incorporated by reference in its entirety.

### TECHNICAL FIELD

The field of art to which this invention generally pertains is food technology, and specifically the use of flavors in food technology.

### BACKGROUND

Traditionally, it has been difficult to predict performance of flavors in flavor encapsulation technologies and target food applications. This has particularly shown itself in the area of maximum flavor load and flavor retention in encapsulation technologies, for example. In the past, the prediction of flavor properties has been based on such things as non-specific solubility data in water or oil, for example. However, this can be difficult to apply to complex blends of flavor components or extracts. Another approach is to use Hansen solubility parameters (Hansen Solubility Parameters, C. M. Hansen, 2^{nd} edition, CRC Press, 2007, 519 p.). Solubility parameters are typically determined for individual flavor components or solvents but need to be calculated for complex flavors based on data. This can be cumbersome for many flavors which can contain numerous components, assuming all the data is available. The data may not be available, for example, for complex natural extracts. Thus, solubility parameters in addition to being labor and time intensive still represent no more than an approximation.

The embodiments described herein address these challenges.

### BRIEF SUMMARY

A method of predicting the performance of individual flavor components in a particular application is described including quantifying the polarity of the flavor component by measuring the dielectric constant of the flavor component, and relating that polarity to flavor component performance in that particular application.

Additional embodiments include: the method described above where the polarity is related to flavor loading, effective encapsulation by extrusion or spray drying, solubility or co-solubility in one or more solvents, and/or coacervation; the method described above where the polarity is related to use of flavor components in food applications; the method described above where the polarity is related to the compatibility and co-solubility of individual flavor components, complex natural extracts, solvents, and/or emulsifiers; and the method described above where the polarity is related to flavor retention, caking, extraction of bioactives, yield, beverage cloudiness, emulsion stability, and/or sensory impact of flavors.

These and additional embodiments are further described below.

### BRIEF DESCRIPTION OF THE DRAWINGS

The FIGURE demonstrates dispersions of flavors of various polarity.

### DETAILED DESCRIPTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As stated above, currently the prediction of flavor properties is typically based on non-specific solubility data in water or oil. There is a large spectrum of solvents and flavors that fall within that range of solubility, some flavors, for example, being partially oil or water soluble with solubility level limits. This evaluation is difficult to apply to complex blends of flavor components or extracts. Another approach is to use Hansen solubility parameters that include polarity as one of the factors. Hansen solubility parameters are determined for individual components and need to be calculated for complex flavors based on this data. This is cumbersome for many flavors often containing over 20 different components, assuming all the data is available. It is also difficult to apply to natural extracts of unknown compositions. Also, the solubility parameters are determined at a specific temperature and may not be accurate at other temperatures. Thus, Hansen solubility parameters are an approximation and are time consuming. Measurement of dielectric constant of a flavor as described herein is a direct method that takes no longer than a few minutes, including equilibration and clean up, thus representing an express analytical method.

As described herein, flavor polarity can be effectively quantified with dielectric constant as measured by conventional dielectric constant meters. Flavor polarity is related to: solubility of flavors in oil, water or other solvents; effective encapsulation of flavors by, for example, melt extrusion, spray-drying, coacervation; functionality of flavors in applications. Polarity of individual flavor components, solvents, and emulsifiers can predict their co-solubility. It can also serve as a quality control method for the ingredients.

Type and solubility of flavors were found to play a significant role in food applications and encapsulation of flavors, for example, by melt extrusion, spray-drying, or coacervation. Typically, flavors are characterized by their solubility in water, oil, or water-ethanol blends. There is no effective and rapid analytical method to accurately quantify, measure, or predict flavor character, properties in processing, and functionality. There is no effective analytical method to predict compatibility of individual flavor components, complex natural extracts, solvents, and emulsifiers other than actual testing their co-solubility. Now these components can be accurately characterized by their polarity as quantified by dielectric constant (DC).

What has been found is that flavors, solvents, and emulsifiers can be effectively and analytically characterized by polarity quantified by dielectric constant. Flavors represent a spectrum by polarity, encompassing a range of dielectric constants at 20° C. from about 2 for very non-polar oils and extracts, e.g. citrus oils of low fold, to about 80 for very polar water soluble flavors, for example, containing mostly water. This can be related to complex natural extracts, compounded flavors, processed flavors, or individual flavor components whether natural or artificial. Similarly, solvents used in flavors and combinations of solvents can also be characterized by polarity and dielectric constant. For example, vegetable oils have dielectric constant about 3 on one end of the spectrum while water has DC about 80 on other end. Co-solubility of flavor components and solvents can be predicted by similarity in polarity as measured by dielectric constant. It has also been found that polarity of flavors is important and even critical in flavor encapsulation, for example, by melt extrusion in natural carriers (see copending, common assigned U.S. patent application Ser. No. (V49330) entitled Natural Encapsulation Flavor Products, filed of even date herewith, the disclosure of which is herein incorporated by reference in its entirety).

Polarity of flavors has an effect on flavor-matrix interaction, which, in turn, determines maximum flavor load that can be achieved in the encapsulation. This defines flavor impact in application and cost in use (or cost of impact) of the encapsulation composition. It has also been found that flavor polarity is also directly related to functionality in applications. For example, more polar flavors could form less cloudy emulsions in beverage applications, being more compatible with water. Now this can be predicted and flavors could be formulated accordingly. Now flavors can be formulated effectively by their polarity, using dielectric constant as a guide. Co-solubility or compatibility of complex flavors can be predicted, individual flavor components, and solvents for optimal performance in processing can be predicted, e.g. in flavor encapsulation, or applications, e.g. in beverages.

Polar solvents such as water heat up in a microwave much faster than non-polar oils. Flavors heat up somewhere in between depending on their polarity. Dipolar moment of molecules of various liquids is different and can be a measure of polarity of liquids. The dipolar moment is responsible for interaction of molecules in a liquid with the electromagnetic field of a microwave oven. One measure of dipolar moment is dielectric constant. Thus, dielectric constant is a sound measure of flavor polarity and can predict flavor properties and flavor-matrix interaction whether during processing or in final target applications. An example of one commercially available dielectric constant meter useful with the method described herein is a model BI-870 from Brookhaven Instruments. It can measure dielectric constant not only at 20° C. but in a broad temperature range.

### Example 1

In support of development of flavors, encapsulation technologies, and applications, dielectric constant of key solvents used in flavor formulation (Table 1) were measured and a variety of target flavors (Table 2). This data predicts compatibility and co-solubility of individual flavor components, solvents, and their blends. Dielectric constants of binary blends of components fall in the range between the dielectric constants of individual components. Other examples demonstrate effect of flavor polarity on performance in technologies and applications.

TABLE 1
Polarity of solvents as measured by dielectric constant at about 20° C.

| | Dielectric | |
|---|---|---|
| Solvent | constant | Temperature |
| High oleic sunflower oil | 3.1 | 20.8 |
| Medium chain triglycerides | 3.9 | 22.1 |
| Triacetin | 7.0 | 21.4 |
| Benzyl alcohol | 13.8 | 21.0 |
| Isopropanol | 20.8 | 21.4 |
| Isopropanol:ethanol 1:1 | 24.1 | 20.6 |
| Ethanol (95%) | 28.3 | 22.0 |
| Propylene glycol | 28.7 | 22.1 |
| Glycerin | 46.1 | 22.0 |
| Water deionized | 79.2 | 22.1 |

TABLE 2
Polarity of selected flavor components and compounded flavors as
measured by dielectric constant at about 20° C.

| | | Dielectric | |
|---|---|---|---|
| Flavor | Solvent | constant | Temperature |
| Orange, single fold | none | 2.2 | 22.0 |
| Cheese-Parmesan | 15.4% medium | 3.3 | 20.9 |
| | chain triglycerides | | |
| Diacetyl | none | 4.4 | 21.4 |
| Raspberry | none | 7.6 | 20.4 |
| Vanilla | 88% triacetin | 8.5 | 20.0 |
| Butter | 43% triacetin | 24.3 | 19.6 |

### Example 2

Flavor load was able to be increased in encapsulation of flavors by melt extrusion by reformulating a flavor. Single-fold orange flavor was modified using water soluble isopropyl alcohol that is also co-soluble to some extent with the flavor. Flavor load was increased from 4% typical for non-polar flavors in non-emulsifying matrices to 6% (Table 3). The idea of linking higher flavor polarity in terms of dielectric constant to higher flavor load was clearly demonstrated in subsequent experiments with a number of flavors as flavor load increased from 4% to 6% and to 8% in melt extrusion encapsulation compositions.

TABLE 3
Maximum flavor load with increased flavor polarity.

| | | | Dielectric | Total flavor |
|---|---|---|---|---|
| Flavor part | Solvent | Emulsifier | constant | load, % w/w |
| 50% Orange | 50% MCT | none | 3.2 | 4% unstable |
| 50% Orange | 15% MCT-35% IPA | none | 6.2 | 6% runs well |
| | | | | slight |
| | | | | surface oil |

### Example 3

The FIGURE shows dispersions of three flavors of various polarity. Orange oil (1) (single fold, DC=2.5), raspberry flavor (2) (DC=7.6), and butter flavor (DC=24.3) at 0.5% by weight in water were each homogenized in 99.9 grams of water at 6,000 rpm (revolutions per minute) for one minute. Low polarity orange flavor forms very cloudy dispersion, raspberry shows slight cloudiness and butter flavor is as clear as water. This demonstrates that a flavor can be chosen or formulated in such a way that its polarity controls cloudiness of aqueous dispersion in target application.

Quantifying flavor polarity of flavor components, flavors, and solvents was found to be and expected to be important in a number of technologies. These technologies include melt extrusion, (high flavor load was achieved with high polarity flavors especially in natural matrices), spray drying (flavor retention, caking), extraction of bioactives and flavors (maximizing yield, extracting specific actives). Flavors can be formulated and optimized based on this fundamental property. Flavor polarity can also be important in applications, for example, controlling cloudiness of beverages, emulsion stability in liquid products, flavor stability in microwave heated products. Finally, flavor polarity can be related to sensory impact of flavors. Polarity of flavors is an important characteristic that defines compatibility of flavor components, solubility, and emulsification of flavors in oil, water, or other media. It is important in prediction of flavor matrix interaction and the choice of most effective emulsifier to use for example in melt extrusion. Polarity can predict flavor matrix interaction and help to avoid surprises in process.

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of predicting the performance of individual flavor components in a particular application comprising
quantifying the polarity of the flavor component by measuring the dielectric constant of the flavor component, and
relating that polarity to flavor component performance in that particular application.

2. The method of claim 1, wherein the polarity is related to flavor loading, effective encapsulation by extrusion or spray drying, solubility or co-solubility in one or more solvents, and/or coacervation.

3. The method of claim 1, wherein the polarity is related to use of flavor components in food applications.

4. The method of claim 1, wherein the polarity is related to the compatibility and co-solubility of individual flavor components, complex natural extracts, solvents, and/or emulsifiers.

5. The method of claim 1, wherein the polarity is related to flavor retention, caking, extraction of bioactives, yield, beverage cloudiness, emulsion stability, and/or sensory impact of flavors.
